(19) United States
(12) Patent Application Publication
(10) Pub. No.: US 2025/0346640 A1
(43) Pub. Date: Nov. 13, 2025

(54) WATER-SOLUBLE LEGUME PROTEIN

(71) Applicant: Emsland-Stärke Gesellschaft mit Beschränketer Haftung, Emilichheim (DE)

(72) Inventor: Nico REINS, Emlichheim (DE)

(21) Appl. No.: 18/718,102

(22) PCT Filed: Dec. 12, 2022
(86) PCT No.: PCT/DE2022/100943

§ 371 Date: Jun. 10, 2024

(30) Foreign Application Priority Data
Dec. 10, 2021 (DE) 20 2021 106 752.7

(51) Int. Cl. C07K 14/415; C07K 1/34

(52) U.S. Cl. CPC C07K 14/415; C07K 1/34

(57) ABSTRACT

Large scale produced water-soluble legume proteins and obtained by: Crushing legume seeds, if necessary, degreasing the crushed legume seeds; Mixing the crushed legume seeds with water to produce a legume slurry; Adjusting the pH value of the legume slurry to a pH value between 6.8 and 7.5, preferably between 7.0 and 7.4; Separating starch and fibers by centrifugation or filtration to produce an aqueous protein solution as supernatant; Adjusting the pH value of the separated protein solution to a pH value between 7.2 and 8, Ultrafiltrating the pH-adjusted protein solution; Diafiltrating the ultrafiltration retentate with water with a pH value of 7.5-8.2 to a conductivity of the diafiltrate of no more than 30% of the conductivity of the permeate without diafiltration, i.e., to a conductivity of 1-3 mS/cm; Obtaining the diafiltered ultrafiltration protein retentate; and drying, cooling or freezing the ultrafiltration retentate, and a method for its production.

### TECHNICAL FIELD

The invention relates to water-soluble legume protein and a method for its production.

### BACKGROUND OF THE INVENTION

In the context of this application, legume protein is understood to mean protein mixtures obtained from legume amniotic fluid.

Legume amniotic fluid is the cloudy aqueous solution that remains in solution when crushed legume seeds are slurried with water after mechanical separation of the suspended particles and water-insoluble materials.

There are various production methods for legume proteins, as explained by Joyce in Food Res. Int. 2010, 43, 414-431, Pulse proteins: Processing, characterization, functional properties and applications in food and feed, doi: 10.1016/j.foodres.2009.09.003 or by Barac et. al. in Int. J. Mol. Sci. 2010, 11, 4973-4990, doi: 10.3390/ijms11124973 or by Taherian in Food Res. Int. 2011, 44, 2505-2514, Comparative study of functional properties . . . . These production and extraction methods influence the parameters that are important for protein use: Solubility, emulsifiability, foaming behavior, film-forming behavior, mouthfeel, taste, etc. M. C. Tulbek, R. S. H Lam, et. al. described in “Sustainable Protein Sources” 2017, chapter 9, pages 145-164, https://doi.org/10.1016/B978-0-12-802778-3.00009-3, that highly functional proteins are sought after as emulsifiers, foaming agents, gelling agents and film formers. A. Singhal, A. C. Karaca, R. Tyler and M. Nickerson also published in 2015 a good summary of grain legume proteins in “Grain Legumes”, Chapter 3: “Pulse Proteins: From Processing to Structure-Function Relationships”, doi: 10.5772/61382.

The invention is explained below with reference to peas (Pisum sativum), beans and lentils—but the method is equally suitable for other grain legume seeds, e.g.

### Phaseolus Species (Phaseolus Ssp.):

Lima bean, moon bean (Phaseolus lunatus L.), port. feijão-de-lima.
Tepary bean (Phaseolus acutifolius A. Gray)
Scarlet runner bean (Phaseolus coccineus L.), span. ayocote, port. feijão-da-espanha/feijoca.
Common bean (Phaseolus vulgaris L.): span. frijol, port. Feijão.
Soybean (Glycine max), span. soja, port. soja.
Pigeon pea (Cajanus cajan), span. guandul, port. guandu
Chickpea (Cicer arietinum), span. Garbanzo, port. grãodebico.
Lentil (Lens culinaris), span. Lenteja, port. lentilha.
Field bean (Vicia faba), span. Faba, port. fava

### Vigna Species (Vigna Ssp.)

Cow pea, (Vigna unguiculata), port. feijão-fradinho
Azuki bean, (Vigna angularis).
Mung bean, (Vigna mungo)
   Lupine species (Lupinus ssp.)
White lupine (Lupinus albus).
Andean lupine (Lupinus mutabilis)
Yellow lupine (Lupinus luteus).
Blue lupine (Lupinus angustifolius).
Multi-leaved lupine (Lupinus polyphyllus)

Species of only local importance include the New World jack bean (Canavalia ensiformis L.) and the Old World sword bean (Canavalia gladiata) in some tropical countries. Helmet beans (Lablab purpureus) are grown in Africa, India and some countries in Southeast Asia. The flat pea (Lathyrus sativus) is mainly important in India because it is considered to be very drought tolerant. Ground beans (Macrotolyma geocarpum) are endemic only in West Africa and ripen in the soil substrate in a similar way to peanuts. Other plants include the horse bean (Macrotolyma uniflorum), yam bean (Pachyrhizus erosus), Goa or wing bean (Psophocarpus tetragonobolus) and the tuber bean or African yam bean (Sphenostylis stenocarpa).

Legume seeds here are understood to mean grain legumes such as peas, chickpeas, lentils, beans—such as field beans, mung beans, soybeans as well as lupine seeds and the like.

The fruits of all grain legumes are characterized by a high protein content. This protein is interesting for a wide variety of applications. It is usually desired that the protein behaves as much as possible like animal protein—i.e. it should replace it in recipes-preferably it is whipable, has an emulsifying effect, and forms films and gels. Thanks to these properties, it can replace animal protein as a binding agent (e.g. in meat products), foaming agent in baking processes and in imitation milk that can be whipped by adding legume protein (e.g. like frothable milk or vegetable cream substitute). However, the legume proteins are also in demand in cosmetics and technology. They are also used as adhesives and adhesive raw materials, for example in photoresists or for glue replacement materials, flotation aids, and emulsifiers.

So far, the use of many legume proteins has failed because they still contain by-products—such as flavonoids, aldehydes, ketones and alcohols, which lead to both taste and solubility problems. As a result, the yield and degree of purity of the soluble protein obtainable using technical methods can be improved and the quality of proteins previously obtained from legume seeds often did not meet the requirements of the food industry with regard to film formation, emulsifying ability, whipping ability, and gel-forming ability. Another problem is that some legume seeds have a very high fat content, which influences, among other things, the solubility of the proteins. Therefore, degreasing methods are used for these legume seeds—as is known for soy. The persons skilled in the art are familiar with degreasing the legume seeds with various solvents. Since proteins with high functionality are particularly desired—i.e., those that are little or not denatured and therefore have a high water solubility, the attempts have been made to find industrial methods to achieve these. Unfortunately, it has not yet been possible to obtain legume proteins in a completely water-soluble form in high yield on an industrial scale using a simple method that can be used industrially.

It is therefore the object of the invention to obtain completely water-soluble legume proteins on an industrial scale with high functionality and high yield.

The object is achieved according to the invention by water-soluble legume proteins with the features of claim 1. The invention further relates to a method according to claim 10. The advantageous developments result from the dependent claims.

The water-soluble legume proteins according to the invention can be produced by:
Crushing husked legume seeds,
If necessary, degreasing the crushed legume seeds; Mixing the crushed and optionally degreased legume seeds with water while adjusting the pH value of the legume slurry to a pH value between 6.8 and 7.5 to form a legume slurry;
Separating the slurry into solids and an aqueous protein solution comprising water-soluble proteins by centrifugal forces or filtration-(methods known to those skilled in the art of starch recovery from natural products), wherein filtering also includes gel filtration and/or centrifugal force separation,
Adjusting the pH value of the filtrate or centrifuge supernatant to a pH value between 7.2 and 8.5, preferably 7.5 to 8.3
Ultrafiltrating the protein solution separated in this way;
Diafiltrating the ultrafiltration retentate with water adjusted to a pH value of 7.5-8.2, selected from fresh water and demineralized water until the diafiltrate has a conductivity of less than 30% of the permeate without diafiltration. Here—depending on the starting material—a conductivity of between 1.0 and 3.0 mS/cm is achieved.
Obtaining the diafiltrated ultrafiltration retentate solution with a solid content of >90% protein (according to Kjeldahl).

Purification steps without thermal stress produce a fully soluble, cloudy protein solution as ultrafiltration retentate, which forms smooth films, foams well, emulsifies and can be processed as such or into dried protein. This ultrafiltration retentate can also be used as an aqueous solution or further processed—e.g. broken down into different protein types by fractionated thermal or pH precipitation. It can be added to other foods or cosmetics in dissolved form to give them the desired properties. What is particularly important for quality is diafiltration with pH-adjusted, demineralized water, which removes disruptive ions, oligosaccharides, sugars and amino acids. Common alkaline materials approved for food use can be used as suitable materials for adjusting the pH value of the diafiltration water, for example NaOH, KOH, Ca(OH)₂, NH₄OH, Mg(OH)₂.

The preparation of the ultrafiltration retentate, the solids of which have a protein content of 90% and more, may be followed by a preservation step of the ultrafiltration retentate, selected from: drying, including lyophilization and/or cooling or freezing of the solution or freeze-drying. The ultrafiltration permeate can be used to recover salts, sugars and oligosaccharides, amino acids and small peptides, wherein it can be subjected to reverse osmosis, which only allows salts and ions to permeate and retains carbohydrates and amino acids. This also has the advantage of reducing wastewater pollution.

The gentle treatment of the proteins during extraction results in highly functional, completely water-soluble proteins that can be used for other purposes. This includes further separation of the proteins or direct protein processing and marketing—e.g. in beverages.

In the field of proteins, “highly functional” refers to those with high water solubility and water-binding capacity as well as good emulsifying properties.

According to a preferred embodiment, the invention comprises additional features which may be included individually or in various combinations depending on their suitability for a particular application.

It therefore relates to water-soluble legume proteins which can be produced by: Crushing of husked legume seeds, Mixing the crushed, possibly degreased legume seeds with water to produce a legume slurry; Adjusting the pH value of the legume slurry to a pH value between 6.8 and 7.5; Separating the legume slurry by centrifugal force or filters into starch and fibers, e.g. by separators, decanters, centrifuges, hydrocyclones, filter centrifuges or vacuum rotary filters/pressure rotary filters, press filters, filter presses, bag filters, candle filters, sheet filters—as known to the skilled person—and an aqueous protein solution; adjusting the pH value of the protein solution separated in this way to a pH value between 7.2 and 8.5; Ultrafiltrating the pH-adjusted protein solution; Diafiltrating the ultrafiltration retentate with water adjusted to pH 7.5-8.2, selected from fresh water and demineralized water, recovering the diafiltrated ultrafiltration retentate; and drying or cooling or freezing of the ultrafiltration retentate as water-soluble legume proteins in solution or as dry material.

The cooled protein solution can be used as such, but also, for example, for further separation into proteins of different molecular weights. However, separation by fractionating isoelectric precipitation is also possible, since different protein groups have different isoelectric points. As a dry protein, the protein powder can be mixed into food or sold as such—the drying method is important for the functionality of the protein and should be as gentle as possible. The wet or moist UF retentate can be added to viscous products such as ice cream or TVPs, which are sold semi-moist from the refrigerated counter or fresh.

For many applications, the water-soluble legume proteins are converted into a completely water-soluble and storable powder by spray-drying, freeze-drying and lyophilization.

It is useful to treat the aqueous ultrafiltration retentate protein solution with adsorbents for the removal of antinutritive components (lectins, protease inhibitors, phytates, tannins, saponins, alkaloids, aldehydes) and for taste improvement, such as activated carbon, flavonoid-adsorbing resins, silicates and other suitable adsorbents known to the skilled person, in particular to remove colorants and certain flavonoids, undesirable antinutritive substances. Volatile components that negatively affect taste and smell, such as aldehydes, alcohols, ketones (see C. Murat, M.-H. Bard, C. Dhalleine, N. Cayot, J. Food Research 2013, 53, 31-41) can also be removed or at least reduced by vacuum extraction or by adsorption on known adsorbents. Phytate present in the protein solution can, for example, be precipitated and removed in a manner known per se by precipitation with divalent ions, usually calcium or magnesium cations, after the starch/fiber separation.

The water-soluble legume proteins, i.e. the diafiltered ultrafiltration retentate, can also be subjected to a HTST treatment (high-temperature short-term treatment) or another preservative step for shelf life.

The cut-off of the ultrafiltration membrane can be between 1 and 100 kDa, with a compromise between yield and selectivity that is easy for the person skilled in the art to determine. The leaching of salts, sugars, amino acids, and other components is beneficial for protein functionality, as shown in FIG. 13 as a diagram of the viscosity development when heating and cooling protein solutions. The samples are sorted according to their protein content (top left—pea amniotic fluid with a low protein content, bottom right—retentate VCR3, 2BV (protein according to the invention) with the highest protein content). The abbreviation VCR (volume concentration factor) describes the concentration factor of the solution and is calculated from the quotient of volume (feed)/volume (retentate). For a filtration with an inflow volume of 300 L and a retentate of 100 L, VCR=3 would result. The abbreviation BV (batch volume) is a measure of the amount of water added during diafiltration. The BV describes the volume that circulates in the system at a certain time. The designation is given during diafiltration to express how much water has been added. Assume you start diafiltration with a retentate volume of 40 L at this point (1 BV=40 L). Then the indication 2 BV Dia. means that 40 L of water were added twice in the batch. These are laboratory values that show the behavior of the retentate—in the large-scale process, this information is only needed to optimize a continuous process.

In FIG. 13 the presented curves show as follows: The top, solid curve is a pea fruit amniotic fluid UF retentate washed with VCR3, 2BV, in which a clear increase in viscosity can be seen in the temperature range from approx. 70° C., which reaches a plateau at 90° C. The dotted curve underneath is the diafiltered UF retentate, which was also diafiltered with VCR3 with water pH 7.5, but was only concentrated to a factor of 3 with 1 BV. It can be seen that doubling the batch volume (BV) leads to a significant drop in viscosity over time or the achievable activation temperature This can be significant in applications, in which the product is cooked or heated to a higher temperature. The curve below (long dashed line) shows the behavior of the UF retentate that was diafiltered with VCR 4.7—you can see a further clear drop in the viscosity behavior or gelling behavior. The dash-dotted line below is a UF retentate that has only been diafiltered with VCR3, which then shows an even lower tendency to gel. The curve below (short dashed line) shows that a VCR2 results in an even smaller increase in viscosity with temperature and the bottom curve is pea amniotic fluid (long dashed double dotted line) that has not been diafiltered or concentrated. There is almost no influence of the temperature treatment on the viscosity and only a very small increase in viscosity is observed.

The viscosity profiles were recorded as follows: A 15% solution of the product in demineralized water was prepared. In the Anton Paar Physica MCR 301 (standard insert, stirrer ST24-2D, 60 rpm), 35 mL of the solution was added according to the temperature profile (start: 25° C., heating 6.5° C./min, hold at 90° C. for 12 min, cooling at 4.3° C./min, holding at 25° C. for 10 min.

All pea protein samples were prepared with tap water. The method was verified again with demineralized water. No qualitative differences could be found.

The water-soluble legume proteins according to the invention can be used as protein separation starting products and/or feed, in foods for protein supplementation, as emulsifiers, film formers, foam stabilizers, glue and glue starting materials, gelling agents, flocculants, fining agents.

### BRIEF DESCRIPTION OF THE DRAWINGS

The invention is described below with reference to the drawing and examples, to which it is by no means limited. Therein:

FIG. 1 is a schematic representation of the method steps for obtaining a water-soluble legume protein mixture according to Example 1 A;

FIG. 2 shows an SDS PAGE gel of the water-soluble pea protein of the invention according to Example 1 A and commercially available pea proteins;

FIG. 3 shows an SDS PAGE gel of mung bean amniotic fluid;

FIG. 4 shows an SDS PAGE gel of a water-soluble protein from field bean isolate, low molecular weight pea protein and pea protein isolate according to DE 102006050619A1;

FIG. 5 shows an SDS PAGE gel of various materials resulting from the ultrafiltration of field bean and mung bean amniotic fluid, including protein isolates and concentrates;

FIG. 6 shows an SDS-PAGE gel of water-soluble proteins from pea amniotic fluid;

FIG. 7 shows HPLC of mung bean, field bean and pea proteins according to the invention;

FIG. 8 shows HPLC of field bean protein solutions of various pretreatment;

FIG. 9 shows HPLC of mung bean proteins of various pretreatment;

FIG. 10 shows HPLC of pea proteins of various pretreatment;

FIG. 11 shows texture measurements on pea proteins with or without thermal treatment at different pH values;

FIG. 12 shows DSC diagrams for thermally treated and thermally untreated pea proteins. and

FIG. 13 shows viscosity measurements of differently diafiltered pea protein ultrafiltration retentates according to the invention

The invention is explained in more detail below with reference to various grain legumes, in particular mung bean, field bean and pea proteins:

### Example 1: Production of Water-Soluble Pea Protein

### Example 1A

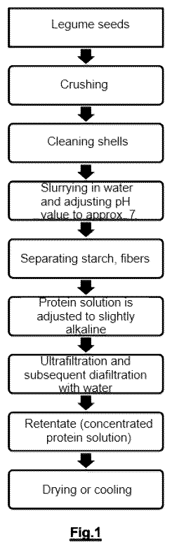

This embodiment of a production method is shown schematically in FIG. 1.

1 kg of dried peas are crushed and mixed with 2.5 kg of water to produce a pea slurry. The pH value of the slurry is adjusted to pH 6.8-7.2 with NaOH. The resulting pea slurry is sieved to remove the shell residues and then starch and fibers are removed using a centrifuge system (hydrocyclone). The overflow/supernatant from the centrifugation/hydrocyclone separation is again subjected to a pH value adjustment to a pH value between 7.5 and 8.5 with NaOH.

The resulting supernatant is now contacted with CaCl₂ to precipitate phytate and with adsorber resin to separate aldehydes, and the insoluble material is centrifuged off. The centrifuge supernatant, i.e. the remaining aqueous protein solution, is now separated via an ultrafiltration system-here with a cut-off of 40 kDa-into an aqueous protein solution as retentate and a salt/amino acid/sugar solution as permeate. The ultrafiltration retentate is now diafiltered/washed with tap water/demineralized water adjusted to pH 7.5-8.0 until a conductivity of the permeate of no more than 30% of the ultrafiltration permeate is achieved. A typical conductivity range is between 1-3 mS/cm. This washes out salts, sugars, glycoproteins and amino acids as well as some of the small proteins with an MW<40 kDa and improves the protein content and purity, i.e. the absence of other molecules. This ultrafiltration retentate protein solution is spray-dried to obtain a light-colored protein powder that is completely soluble in water.

### Example 1B

To produce highly water-soluble pea protein, the dried paler peas were de-husked, crushed and slurried in water. The suspension is subjected to gravity separation (centrifugation) and the supernatant is used as protein-rich amniotic fluid for protein extraction.

The protein-containing solution is centrifuged again, wherein the fine suspended particles are removed from the solution. The purified protein-containing solution is adjusted to a pH value of 7.0 to 8.0 and then ultrafiltrated and diafiltered with demineralized water to an electrical conductivity of 1.5 to 3.0 mS/cm. The protein according to the invention is obtained from the ultrafiltration retentate, while salts, sugars and amino acids remain in the ultrafiltration permeate. For this purpose, the UF retentate is sterilized using HTS and then spray-dried. An analysis of the light-colored protein powder produced in this way revealed:

| | |
|---|---|
| Humidity [%] | 6.7 |
| Protein content [%] | 80.3 |
| Product solubility [%] | 94.7 |
| Protein solubility [%] | 92.3 |
| Fat [%] | 6.8 |
| Ash [%] | 6.4 |

The pea protein according to the invention forms gels (heat- or acid-induced) and has a strong emulsifying effect (see also FIG. 11). With a solubility of 94.7%, this is not a clear solution, but a completely dissolved cloudy solution.

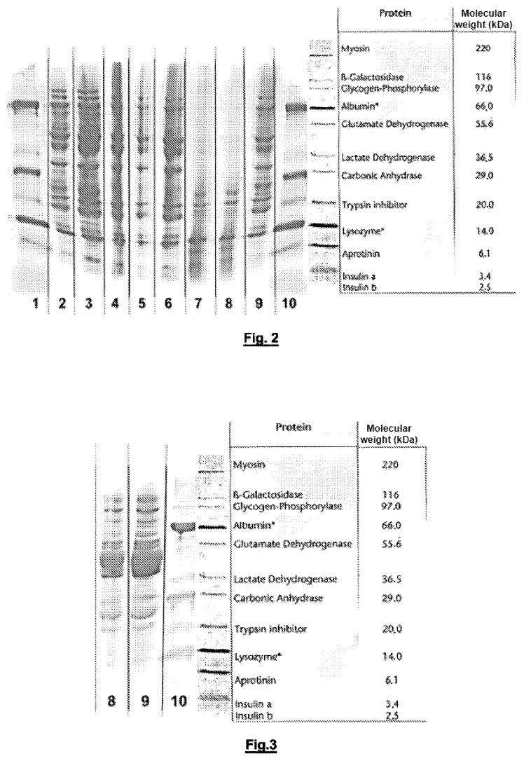

In FIG. 2, SDS-PAGE gels of pea proteins according to the invention are compared with commercially available pea proteins. It can be clearly seen that the pea protein according to the invention (lanes 2 and 3) from various crushed peas has proteins with an MW between about 6 kDa and about 120 kDa. The pea protein Pisane C9 (Cosucra) (lane 4) available on the market contains proteins of higher molecular weight; Nurtralys S 85XF (lane 5), Nutralys S 85 F (lane 6) (Roquette) show clear protein spectrum shifts towards higher molecular weights or lower molecular weights. The pea protein of lane 9 produced by EMSLAND Starch, obtained according to the German patent DE102006050619 B1 by means of isoelectric precipitation and temperature increase, shows proteins of medium and higher molecular weight—a clear shift in the molecular weight ratios of the various pea proteins is visible. However, since SDS-PAGE gels are not a quantitative analysis, but only allow statements to be made about qualitative properties, the information is relative.

Proteins with an MW>120 kDa tend to precipitate out of the aqueous solution and have a less pronounced emulsifying capacity and are therefore less suitable for many applications. The pea protein solution according to example 1—i.e. the ultrafiltrated and water-diafiltered retentate of the ultrafiltration—was separated in a denaturing SDS gel chromatography. It can be clearly seen that no bands for proteins with a molecular weight<10 kDa occur in the protein mixture according to the invention and that proteins with a molecular weight above 150 kDa are also absent.

The pea proteins according to the invention have a molecular weight of 150 kDa to about 14 kDa and have proteins of different molecular weights with centers of gravity around 14, 40 and 97 kDa. In contrast, the comparison product Pisane C9 still contains many proteins with a molecular weight>116 kDa, which interfere with solubility in water. The Nutralys S 85 products also contain these large proteins. In contrast, the Nutralys S85 Plus products have a molecular weight between 30 and about 3 kDa; proteins of higher molecular weights are obviously only present in small quantities.

The protein spectrum of the product according to DE102006050619A1 in turn has a higher proportion of proteins of higher molecular weight.

It is assumed that the proteins with a molecular weight>120 kDa are only incompletely soluble in water; this also applies to Pisane and Nutralys S85. Nutralys S85, on the other hand, suggests that pea proteins with a molecular weight of >30 kDa are missing at the expense of the protein yield—i.e. it is a different fraction of pea proteins. It can therefore be stated that the water-soluble protein mixture according to the invention has a different, new protein spectrum than the products available on the market-which is due to the gentle isolation process according to the invention.

A taste test in the case of the egg white according to the invention left a neutral, gel-like taste or mouthfeel.

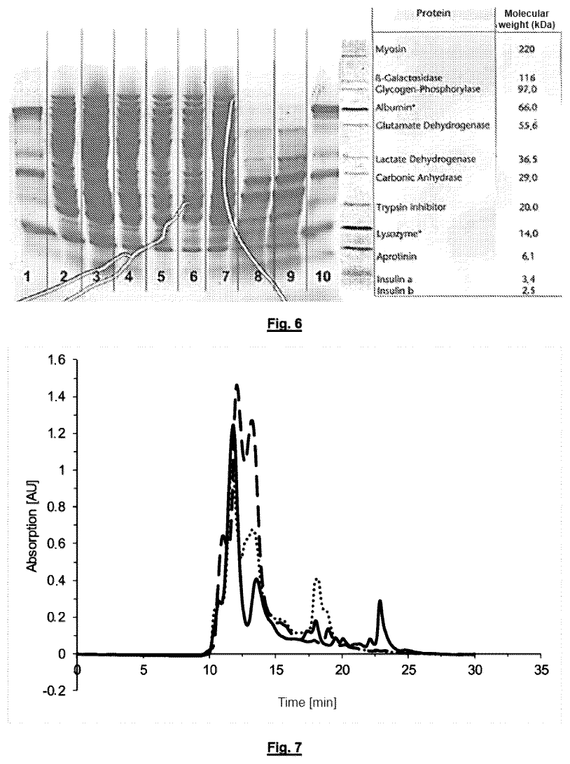

In FIG. 6, a pea protein separation with low thermal stress is examined. For this purpose, the filtrate of the pea slurry was subjected to SDS-PAGE analysis. Lanes 2 and 3 show the filtrate, lane 4 shows the retentate on the UF membrane, not diafiltered, lane 5 shows the diafiltered retentate, lanes 6 and 7 show the produced, thermally untreated product F-1140 according to the invention. It can be clearly seen that in the permeate of the 100 kDa membrane (lane 8), proteins with an MW>30 kDa are only present in very small quantities, while proteins with an MW<40 kDa are predominantly found. Here too, it can be seen that the thermal treatment of the permeate (spray drying) leads to aggregated or coagulated proteins of higher molecular weights (lane 9), while the non-thermally treated permeate in lane 8 (high dilution) hardly shows such proteins. It should be noted that the detection limits for SDS-PAGE are approx. 1 g/I and the dilution of the sample results in a gel, the bands of which are only just above the detection limit.

The analysis conditions of FIG. 6 can be seen in the following table

| Track | Weighing | Dilution | Feed quantity | |
|---|---|---|---|---|
| No. | [g] | [μL] | [μL] | Sample description |
| 1 | — | 0 | 10 | Marker |
| 2 | 50 | 200 | 10 | Inlet membrane |
| 3 | | | 20 | |
| 4 | 20 | 200 | 10 | Retentate VCR 3 |
| 5 | 20 | 300 | 10 | Retentate 2BV Dia |
| 6 | 2 | 300 | 10 | Pea protein according to |
| 7 | | | 20 | the invention |
| 8 | 100 | 0 | 20 | Permeate 100K liquid |
| 9 | 5 | 0 | 20 | Permeate 100K spray-dried |
| 10 | — | 0 | 10 | Marker |

### Example 2a: Production of Mung Bean Protein

1 kg of dried mung beans are crushed and mixed with 3 kg of water to produce a mung bean slurry. The pH value of the slurry is then adjusted to pH 6.8-7.2 with NaOH. The mung bean slurry is sieved to remove the shell residues and then starch and fibers are removed using a centrifuge system. The supernatant from the centrifugation is again subjected to a pH value adjustment to a pH value between 7.5 and 8.2.

The resulting supernatant is treated with CaCO₃ to precipitate the phytate and with adsorber resin and the precipitated solids are separated by centrifugation. The remaining aqueous protein solution is now separated via an ultrafiltration system—here with a cut-off of 15 kDa—into a protein concentrate as a retentate and a salt/amino acid/sugar solution with some smaller proteins (MG<15 kDa). The ultrafiltration retentate is washed with demineralized water at pH=8 until a conductivity of less than 2 mS/cm is achieved. This ultrafiltration retentate solution is now spray-dried to a light-colored protein powder that is completely soluble in water.

An SDS-PAGE gel of the resulting protein mixture is shown in FIG. 5 in lanes 8 and 9. As a result, most water-soluble proteins have a molecular weight between 40 kDa and 55 kDa, while the proteins of other molecular weights occur less frequently.

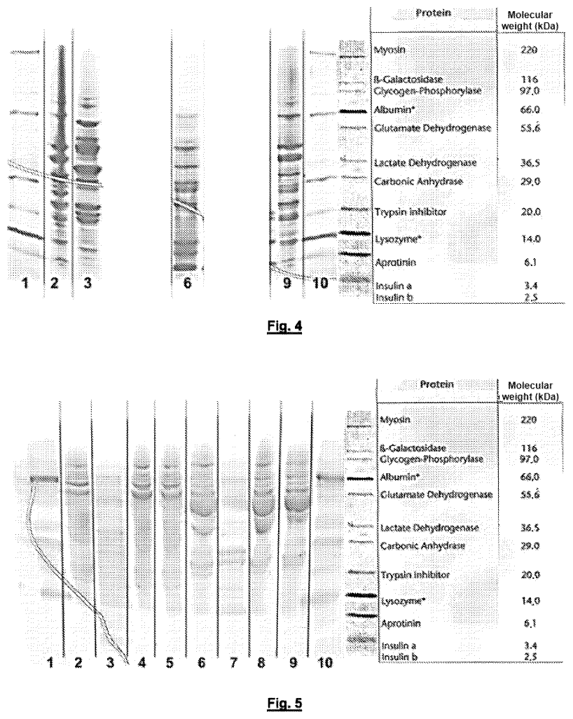

Also from FIG. 4, which shows a comparison between mung bean proteins produced according to the invention (lane 2), field bean proteins (lane 3), and low molecular weight pea protein produced according to DE202021102596.4 with thermal precipitation in lane 6 and in lane 9, a pea protein after isoelectric precipitation according to DE102006050619B1, it is obvious that proteins of specific molecular size ranges are obtained by precipitation or ultrafiltration, which are commercially usable depending on their properties. The HPLC of mung bean proteins and pea proteins as well as field bean proteins in FIG. 6 also shows this behavior: Mung bean proteins (producible according to example 2B) and field bean proteins (producible according to example 4B) have larger amounts of proteins in the retention time range of 10-15 min, while pea proteins isolated via membrane according to the invention have a proportionally smaller amount of these proteins compared to proteins with a retention time of 15-25 min.

### Example 2B: Production of Highly Water-Soluble Mung Bean Protein

To produce highly water-soluble mung bean protein, the dried mung beans were de-husked, crushed and slurried in water. The suspension is subjected to gravity separation (centrifugation) and the supernatant is used as protein-rich amniotic fluid for protein extraction.

The protein-containing solution is adjusted to a pH value of 7.0 to 8.0 and centrifuged again to remove fine suspended particles from the solution. The purified protein-containing solution is ultrafiltrated and diafiltered with demineralized water to an electrical conductivity of 1.5 to 3.0 mS/cm. The protein according to the invention is obtained in the ultrafiltration retentate, while salts, sugars and amino acids remain in the ultrafiltration permeate.

The ultrafiltration retentate with subsequent spray drying exhibited:

| | |
|---|---|
| Humidity [%] | 4.8 |
| Protein content [%] | 83.2 |
| Product solubility [%] | 99.0 |
| Protein solubility [%] | 98.3 |

The mung bean protein according to the invention forms gels (heat- or acid-induced) and has a strong emulsifying effect.

### Example 3: Production of Lentil Protein

1 kg of dried, crushed and degreased lentils are mixed with 2.6 kg of water to produce a lentil slurry. The pH value of the slurry is then adjusted to pH 6.8-7.2 with NaOH. The lentil slurry is sieved to remove the shell residues and then starch and fibers are removed using a centrifuge system. The supernatant from the centrifugation is again subjected to a pH value adjustment with KOH to a pH value between 7.5 and 8.2.

The resulting supernatant is contacted with CaCl₂ to precipitate the phytate and with aldehyde adsorber resin and the precipitated solids are separated by centrifugation. The remaining aqueous protein solution is now separated via an ultrafiltration system-here with a cut-off of 70 kDa-into a protein solution as a retentate and a salt/amino acid/sugar solution. The ultrafiltration retentate is diafiltered with tap water of pH 7 until the conductivity of the permeate is about 20% of the conductivity of the protein solution used in the ultrafiltration. This ultra filtration retentate is now treated with activated carbon for adsorption of dyes and off-flavors and then lyophilized to a light protein powder that is completely soluble in water.

### Example 4a: Production of Field Bean Protein

1 kg of dried field beans are crushed and mixed with 3 kg of water to produce a field bean slurry. The pH value of the slurry is then adjusted to pH 6.8-7.2 with NaOH. The field bean slurry is sieved to remove the shell residues and then starch and fibers are removed using a centrifuge system. The supernatant from the centrifugation is again subjected to a pH value adjustment to a pH value between 6.8 and 8.3.

The resulting supernatant is contacted with CaCl₂ to precipitate the phytate and with adsorber resin and the precipitated solids are separated by centrifugation. The remaining aqueous protein solution is now separated via an ultrafiltration system, here with a cut-off of 15 kDa, into a protein concentrate as a retentate and a salt/amino acid/sugar solution. The ultrafiltration retentate is washed with fresh water adjusted to pH 7.8 with NH₄OH until a conductivity of less than 2 mS/cm is achieved. This ultrafiltration retentate solution is now spray-dried to a light-colored protein powder that is completely soluble in water

### Example 4B: Production of Highly Water-Soluble Field Bean Protein

To produce highly water-soluble field bean protein, the dried field beans were de-husked, crushed and slurried in water. The suspension is subjected to gravity separation (centrifugation) and the supernatant is used as protein-rich amniotic fluid for protein extraction.

The protein-containing solution is centrifuged again, wherein the fine suspended particles are removed from the solution. The purified protein-containing solution is adjusted to a pH value of 7.0 to 8.0 and then ultrafiltrated and diafiltered with demineralized water to an electrical conductivity of 1.5 to 3.0 mS/cm. The protein according to the invention is obtained in the ultrafiltration retentate, while salts, sugars and amino acids remain in the ultrafiltration permeate. The ultrafiltration retentate, obtained by subsequent spray-drying of the UF retentate, exhibited:

| | |
|---|---|
| Humidity [%] | 4.9 |
| Protein content [%] | 88.0 |
| Product solubility [%] | 100.0 |
| Protein solubility [%] | 100.0 |

The field bean protein according to the invention forms gels (heat- or acid-induced) and has a strong emulsifying effect.

An HPLC analysis of field bean products that occur during the production of the protein mixture according to the invention is shown in FIG. 8. The filtered and centrifuged amniotic fluid is clearly visible (dotted line). The permeate of the UF is shown as the solid line, wherein the ratio of peaks between the retention time of 10-15 min and 15-25 min shifts drastically towards the proteins with the longer retention times-those with shorter retention times are no longer detectable. In contrast, the field bean isolate-dashed line-mainly contains proteins with a molecular weight corresponding to a retention time of 10-15 min, which were already predominantly present in the filtrate. HPLC thus shows the successful separation of proteins with retention times of >18 min using UF.

Legume proteins according to the invention as well as intermediate and by-products of protein extraction from the field beans and mung beans were analyzed by SDS-PAGE (FIG. 5). It was found that the amniotic fluid of these beans (lane 2=field bean amniotic fluid; lane 6=mung bean amniotic fluid) after diafiltration according to the invention over a UF membrane yielded UF retentates (lanes 4 and 5 for field beans and lanes 8 and 9 for mung beans) which did not show any bands below approx. 40 kDa. The UF permeate of a 100 kDa ultrafiltration membrane showed (lane 3 for field beans and lane 7 for mung beans) only very weak bands especially in the range of >40 kDa.

FIG. 7 shows an HPLC of mung bean protein isolate (dashed line, producible according to Example 2B), field bean isolate (solid line, producible according to Example 4B) and pea protein according to the invention (dotted line). All proteins were produced according to the method of the invention using an UF membrane of 100 kDa. It can be clearly seen that the mung bean and field bean isolate have a lot of protein with a molecular weight of a retention time of approx. 10-15 min, while the pea protein UF retentate also has proteins with a retention time of 15-20 min.

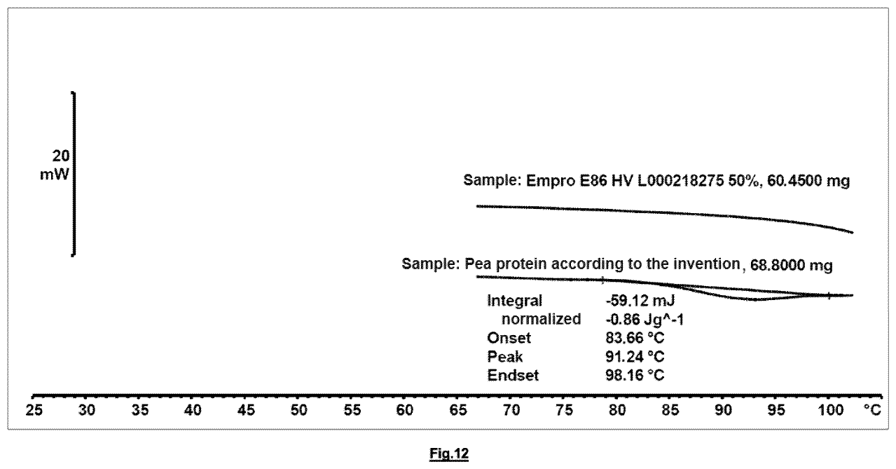

An analysis of the pea protein production using HPLC in FIG. 10 also showed that in the pea protein according to the invention (dotted line) only proteins with retention peaks in the range of 9-19 min occur, while the ultrafiltration separates the proteins with the retention times above this. The permeate (solid line) only shows proteins in the range of 18-26 min. This shows the separation efficiency of the UF membrane, since the pea protein according to the invention (dotted line), originating from the UF retentate, mainly contains proteins up to a retention time of 20 min.

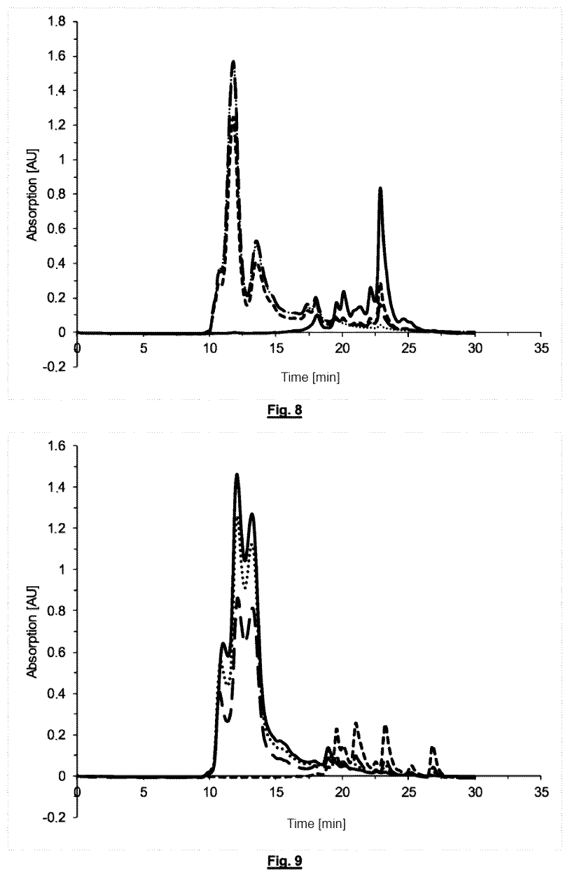

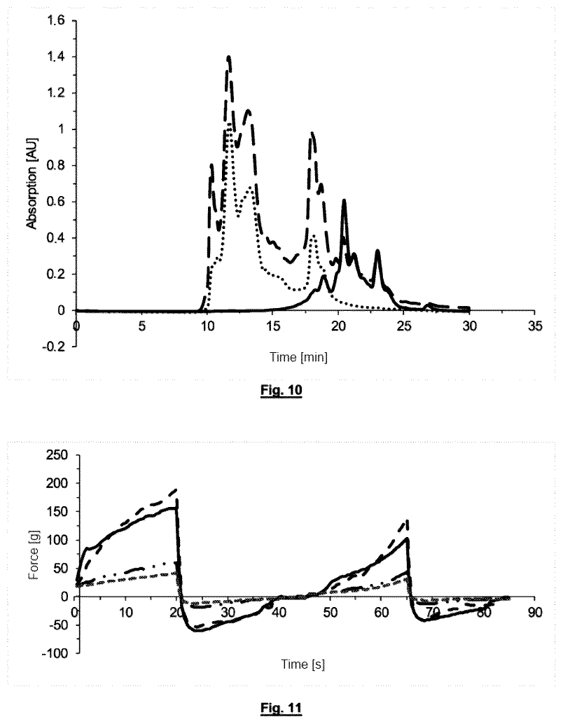

FIG. 9 shows an HPLC diagram for mung bean protein similar to that in FIG. 10. Here, too, there is a clear shift in peak intensity towards 10-15 min in the UF retentate (dotted line), while the permeate (short dashed line) essentially only contains proteins with a retention time of 18-27 min. The HPLC diagram of the centrifuged mung bean amniotic fluid (long dashed line) still contains clear bands of 18-27 min, while the peak at 10-15 min is smaller in relation to the other peaks. The permeate (short dashed line) mainly contains proteins in the molecular weight range of 20-30 min, which have been almost completely separated in the protein according to the invention.

FIG. 8 shows an HPLC diagram for field bean protein similar to that in FIG. 10. Here, too, there is a clear shift in the peak intensity of the field bean filtrate (dotted line) towards 10-15 min in the retentate (dash-dotted line), while the permeate (solid line) essentially only contains proteins with a retention time of 18-27 min.

FIG. 7 shows a comparison of the HPLC diagrams of pea (dotted line), field bean (solid line) and mung bean (dashed line) protein isolates according to the invention. It can be seen that all these legumes have similar protein peaks, wherein the peas seem to have relatively less protein in the peak between 10-15 min per protein 17-25 min than field beans or mung beans.

FIG. 11 shows the tests on the gel formation behavior. The legume proteins according to the invention form the thermally—but also via the pH value-activatable gels that are elastic for a long time. The properties of the gels were examined by means of texture analysis with the TA XT plus Texture Analyzer using the plunger (SMS P 05) (path: 20 mm, forward, test and return speed: 1.0 mm/sec; release force: 20 g) at room temperature.
Preparation of 2 samples: preparing protein (6 g or 12 g product) and adding demineralized H₂O (34 g or 68 g), stirring until the protein is dissolved
Pouring 30 mL of the sample solution into an Anton Paar rheometer with metal cylinder with hole (H-CC27-D).
Boiling the sample solution: starting temperature of 25° C., heating phase: heating to 90° C. at a heating rate of 6.5° C./min, holding time at 90° C. for 15 min, cooling phase: cooling to 25° C. at a cooling rate of 4.0° C., holding time at 25° C. for 10 min
Storing boiled sample solution for 24 h at room temperature
Texture analysis with the TA XT plus Texture Analyzer

In the measurement method, a plunger is slowly pressed into the prepared sample solution, which corresponds to the first peak. When moving the plunger into the gel, a force must be applied until the plunger has completely penetrated the gel. The negative force then corresponds to the retraction of the plunger and the elastic tightening of the gel. The process is then repeated and the plunger penetrates the gel a second time. The maximum force is usually lower in the second process, since the gel strength is still impaired by the first process. The more similar the peaks are, the greater the elasticity of the gel. It can be clearly seen that different forces are required to penetrate the gel—wherein the gel strength is strongly dependent on the pH value. While the gel strength is increased by acidification to a pH value of 6, it decreases by further reducing the pH value. This can be clearly seen from FIG. 11: the solid line means—pea protein solution according to the invention without pH reduction (pH=7.6); dashed line—pH=6; dotted line—pH=5 and dotted line—pH=4.

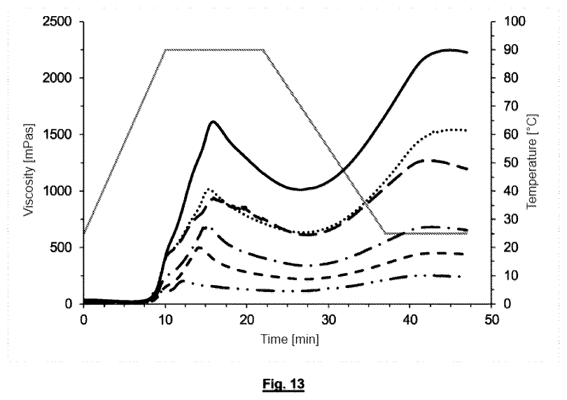

A differential scanning calorimetry (DSC) analysis (FIG. 12) showed, as already known from SDS gels, that a thermal treatment (HTST) altered the proteins. The DSC measurements were carried out to investigate the thermal properties of the protein, which allows conclusions to be drawn about the denaturation state of the protein. Dry or liquid samples are heated and their heat absorption is measured over the tested temperature range. For the measurement, a 50% solution of the protein according to the invention without sterilization and of denatured EMPRO E86 HV, a commercially available, thermally treated, pasteurized protein adjusted to a pH value of 8.2-9.2 before spray drying according to DE102006050619A1, was prepared in demineralized water and filled into a 100 μI aluminium crucible. The measurement was carried out on a DSC+Star^{e} System from Mettler Toledo® in a nitrogen atmosphere. A temperature range of 25-105° C. with a heating rate of 10° C./min was investigated. FIG. 12 shows that the pea protein according to the invention absorbs heat from a temperature of approx. 84° C., which corresponds to the start of denaturation (Tₒₙₛₑₜ). Heat absorption ends at approx. 98° C., resulting in a denaturation peak temperature (T_{dPeak}) of approx. 91° C. This shows that the pea protein according to the invention has not previously been heat-treated and therefore still contains native proteins. In comparison, the denatured sample of the Emsland starch pea protein Empro E 86 HV (coagulation by means of pH adjustment and heat, as well as pasteurization) shows no heat absorption, since this protein is already thermally denatured. A higher denaturation temperature is usually due to larger and more complex proteins.

Further application examples are given below, which show possible uses of the proteins according to the invention-further applications are obvious to the person skilled in the art.

### Example 5: Protein-Enriched Pasta

| Ingredients | Concentration [%] |
|---|---|
| Clean label pea starch Empure EJEL 100 | 34 |
| Native pea starch | 30 |
| Pea protein according to the invention | 25 |
| Pea fiber Emfibre EF 200 | 10 |
| Guar gum flour | 1 |

### Production:

1. Mixing all dry ingredients well
2. Adding cold tap water until a humidity of approx. 30-34% has been reached
3. Mixing/kneading with the Häussler® PN300 VXS noodle machine for approx. 15 min
4. Combining and shaping with a single screw extruder
5. Drying up to a moisture content<13%

By using the pea protein according to the invention, a gluten-free and protein-enriched pasta could be produced. Further advantages of using the pea protein according to the invention are a lighter color than the potato protein Empro K, which had a darker color, and a more pleasant, less bitter taste than the potato protein Empro K and the pea protein Empro E 86 HV (a denatured, temperature-treated pea protein from EMSLAND STÄRKE). By using different proteins in combination, different textures/consistencies can be achieved.

### Example 6: Slicable, Vegan Imitation Cheese

| Ingredients | Concentration [%] |
|---|---|
| Water | 45.2 |
| Coconut fat | 19.4 |
| Oxidized potato starch Emox C 70 S ® (E 1404) | 19.3 |
| Pea protein according to the invention | 7.8 |
| Cross-linked potato starch Emflo 991 ® | 5.02 |
| n-OSA potato starch Emfix K 02 ® (E 1450) | 1.9 |
| Salt | 0.9 |
| Flavor* | 3.3 |
| Citric acid | 0.10 |
| β- Carotene | 0.05 |

*Various tastes available, e.g., “cheddar cheese taste”

1. Mixing all dry substances
2. Water and fat were placed in a heatable stirring vessel and heated to 50° C. at low speed (300 rpm) to melt the fat
3. Adding the dry mixture and heating to 80-85° C. while stirring (approx. 500 rpm), holding the temperature for 5 min
4. Pouring into molds and storing in a cool place at 6-8° C. for 5 days

By using the pea protein according to the invention, it was possible to produce a slicable imitation cheese, the protein content of which is much closer to that of milk cheese.

### Example 7: Vegan Ice Cream

| Ingredients | Concentration [%] |
|---|---|
| Water | 42.3 |
| Vegan milk | 35.0 |
| Sugar | 8.0 |
| Pea protein according to the invention | 5.0 |
| Coconut fat | 4.0 |
| Glucose syrup | 3.0 |
| Cocoa powder | 1.9 |
| Stabilizer mixture | 0.5 |
| Salt | 0.1 |
| Flavoring agent | n.d. |

1. Putting the water and milk in a Thermomix®
2. Mixing the dry substances and stirring in at level 4, reducing to level 3.5 after one minute
3. Heating the glucose syrup in the microwave (2 min, 800 W)
4. Melting the coconut fat with the previously heated glucose syrup in a saucepan on the stove and stirring until completely dissolved
5. Pouring the mixture into the pan, stirring for 1 min
6. Heating up to 90° C. and holding this temperature for 10 min
7. Cooling the mixture to 15° C. using a water bath
8. Adding flavors, storing overnight in a cool place (6° C.)
9. Processing in the ice cream machine

By using the pea protein according to the invention, it was possible to produce a vegan ice cream which had a creamy mouthfeel and a pleasant, slightly nutty taste. The ice cream produced in this way is also protein-enriched.

### Example 8: Vegan Burger

| Ingredients | Concentration [%] |
|---|---|
| Water | 59.4 |
| TVP based on pea protein | 21.0 |
| Oil | 10.0 |
| Pea protein according to the invention | 5.0 |
| Psyllium husks | 1.3 |
| Clean label pea starch Empure ES 300 | 1.0 |
| Methylcellulose | 1.0 |
| Guar gum | 1.0 |
| Modified potato starch Emfix K 02 (E 1450) | 0.2 |
| Sugar color | 0.1 |
| Flavorings and colorants | n.d. |

1. Hydrogenating the TVPs
2. Crushing the TVPs if required
3. Adding all other ingredients and mixing all ingredients
4. Shaping the burger patties

The shaped burger patties can be fried directly or frozen first and prepared at a later time. Even after the freeze-frying process, the functionality of the protein according to the invention is retained. The protein according to the invention helps to improve the product binding and firmness of the patties.

### Example 9: Vegan Sausage

| Ingredients | Concentration [%] |
|---|---|
| Water | 63.0 |
| Spirit vinegar | 0.5 |
| HME product (wet extrudate based on pea protein) | 10 |
| Pea protein according to the invention | 6.0 |
| Hydroxypropylated cooked starch based on peas - | 4.0 |
| Emden ESH 15 ® | |
| Psyllium husks | 8.0 |
| Carrageenan | 1.0 |
| Salt | 0.8 |
| Flavor | n.d. |
| Colorant | n.d. |

1. Mixing all dry ingredients
2. Crushing wet extrudate
3. Adding the mixed ingredients to the water and vinegar and mixing everything together
4. Adding crushed wet extrudate and mixing
5. Fill the mixture into a sausage husk
6. Heating the vegan sausage in a convection oven at 90° C. for 1.5 h
7. Cooling to 7° C. and storing at this temperature for approx. 48 h

The pea protein according to the invention was used to produce a plant-based sausage. By using the protein according to the invention, a greater protein enrichment can be achieved than with other pea proteins, since it has a lower viscosity and thus offers advantages in terms of processability.

The description of the disclosure is merely exemplary in nature and therefore examples that do not deviate from the content of the disclosure are intended to be within the scope of the disclosure. Such examples are not to be regarded as a deviation from the spirit and scope of the disclosure. The broad teachings of disclosure can be implemented in a variety of forms. Therefore, although this disclosure contains certain examples, the true scope of the disclosure should not be limited thereto, since other modifications will become apparent upon study of the drawing, the description and the following claims.

1-10. (canceled)

11. A water-soluble legume proteins, produced by:
crushing legume seeds,
if necessary, degreasing the crushed legume seeds;
mixing the crushed legume seeds with water to produce a legume slurry;
adjusting the pH value of the legume slurry to a pH value between 6.8 and 7.5, preferably between 7.0 and 7.4;
separating starch and fibers by centrifugation or filtration to produce an aqueous protein solution as supernatant;
adjusting the pH value of the separated protein solution to a pH value between 7.2 and 8.5, preferably 7.5-8.3;
ultrafiltrating the pH-adjusted protein solution;
diafiltrating the ultrafiltration retentate with water, selected from fresh water and demineralized water, with a pH value of 7.5-8.2 to a conductivity of the diafiltrate of no more than 30% of the conductivity of the permeate without diafiltration, i.e. to a conductivity of 1-3 mS/cm;
obtaining the diafiltered ultrafiltration protein retentate; and
drying, cooling or freezing the ultrafiltration retentate.

12. The water-soluble legume proteins according to claim 11, wherein the drying is selected from spray-drying, freeze-drying, lyophilization.

13. The water-soluble legume proteins according to claim 11, wherein the aqueous ultrafiltration retentate is treated with an adsorbent selected from activated carbon, silicates and adsorbent resins.

14. The water-soluble legume proteins according to claim 11, wherein a phytate precipitation is carried out after the separation of starch and fibers in the protein solution.

15. The water-soluble legume proteins according to claim 11, wherein the legumes are selected from beans, including mung beans, peas, chickpeas, lupins, lentils.

16. The water-soluble legume proteins according to claim 11, wherein the ultrafiltration retentate is temperature-treated, selected from UHT, HTST.

17. The water-soluble legume proteins according to claim 11, wherein the cut-off of the ultrafiltration membrane is between 5 and 100 kDa.

18. The water-soluble legume proteins according to claim 11, which are starting products and/or finished products for animal feed, and/or emulsifiers, and/or film formers, and/or foam stabilizers and/or foodstuffs or additives for human and animal nutrition, gluten starting material or fining agents for fruit juices and beverages produced therefrom.

19. A method for producing soluble legume proteins, comprising the steps of:
pre-sowing crushed legume seeds,
if necessary, degreasing the crushed legume seeds;
mixing the crushed legume seeds with water to produce a legume slurry;
adjusting the pH value of the legume slurry to a pH value between 6.8 and 7.5, preferably between 7.0 and 7.4;
separating starch and fibers by centrifugation or filtration to produce an aqueous protein solution as supernatant;
adjusting the pH value of the aqueous protein solution to a pH value between 7.2 and 8.5; preferably 7.5-8.3;
ultrafiltrating the pH-adjusted protein solution;
diafiltrating the ultrafiltration retentate with water, selected from fresh water and demineralized water, with a pH value of 7.5-8.2 up to a conductivity of the diafiltrate of no more than 30% of the conductivity of the permeate without diafiltration;
obtaining the diafiltered ultrafiltration protein retentate;
if necessary, HTST treatment of the UF retentate and
drying, cooling or freezing the ultrafiltration retentate.

20. The method according to claim 19, wherein a UF membrane with a cut-off of 10-100 kDa is used.
